(19) United States
(12) Patent Application Publication
(10) Pub. No.: US 2026/0137633 A1
(43) Pub. Date: May 21, 2026

(54) DEXMEDETOMIDINE MICRONEEDLE PATCH, PREPARATION METHOD AND APPLICATION THEREOF

(71) Applicant: GUANGZHOU NOVAKEN PHARM CO., LTD., Guangzhou (CN)

(72) Inventors: Beibei Yang, Guangzhou (CN); Yu Tang, Guangzhou (CN); Langkun Liao, Guangzhou (CN); Dan Yang, Guangzhou (CN); Hualiang Liu, Guangzhou (CN); Chuanbin Wu, Guangzhou (CN)

(21) Appl. No.: 19/303,705

(22) Filed: Aug. 19, 2025

(30) Foreign Application Priority Data
Nov. 21, 2024 (CN) 202411676053.5

(51) Int. Cl. A61K 9/70; A61K 9/00; A61K 31/4174; A61K 47/32; A61K 47/38

(52) U.S. Cl. CPC A61K 9/7084; A61K 9/0021; A61K 31/4174; A61K 47/32; A61K 47/38

(57) ABSTRACT

The present disclosure relates to a dexmedetomidine microneedle patch, a method for preparing the same, and application thereof. The dexmedetomidine microneedle patch includes a substrate and a needle body disposed on a surface of the substrate, wherein the needle body includes dexmedetomidine hydrochloride, a matrix material, and a permeation enhancer. The permeation enhancer includes one or more of dodecyl-β-D-maltoside, ethylenediaminetetraacetic acid, and tetrahydropiperine.

### CROSS-REFERENCE TO RELATED APPLICATIONS

This application claims priority to Chinese patent application No. 202411676053.5, filed on Nov. 21, 2024, the content of which is hereby incorporated by reference in its entirety.

### TECHNICAL FIELD

The present disclosure relates to the technical field of pharmaceutical preparations, and in particular to a dexmedetomidine microneedle patch, a method for preparing the same, and application thereof.

### BACKGROUND

Insomnia is a common sleep disorder, characterized by difficulty falling asleep, difficulty maintaining sleep, or being unable to fall asleep again after waking up early, accompanied by daytime dysfunction, etc. Insomnia patients often show symptoms of inattention, memory loss, slow reaction, and emotional instability, which can seriously affect work efficiency and quality of life. Insomnia is closely related to psychological disorders such as anxiety and depression, and can also lead to decreased immunity and a variety of physical health problems. Studies have shown that the incidence of insomnia is high worldwide, with a prevalence of about 10% to 30% in the general population, and with an even higher prevalence in specific populations, such as postoperative patients, the elderly, women, and people with chronic diseases or mental disorders. With the increase of age, the incidence of insomnia increases significantly, especially in people over 50 years old. For postoperative patients, insomnia can have a significant negative impact on the rehabilitation process, including delaying the rehabilitation process, aggravating postoperative pain, increasing the risk of postoperative complications, prolonging hospitalization, increasing medical costs, etc. Conventional drugs for treating insomnia include benzodiazepines, barbiturates, melatonin, etc. These drugs have varying degrees of addiction, drug tolerance, next-day residual effects, impaired cognitive function, or other adverse reactions.

Dexmedetomidine is a highly selective α2 adrenergic receptor agonist that can produce corresponding hypnotic effects by acting on α2 receptors in the central nervous system and peripheral nervous system. Compared with other drugs for treating insomnia, dexmedetomidine has unique advantages, including generation of a similar hypnotic effect to natural human sleep, no respiratory depressant effect, protection of the upper respiratory tract, reducing postoperative delirium, and low addiction and dependence, which is particularly important for patients who use sleep aids for a long time.

A method for preparing a soluble microneedle by combining dextran and dexmedetomidine hydrochloride is proposed for preoperative sedation in children. However, compared with preoperative sedation in children, the soluble microneedle is required to have a higher cumulative drug permeation in the treatment of insomnia to achieve the purpose of shortening the onset time of sleep-inducing effect and prolonging the duration of sleep-inducing effect, but the composition of dextran and dexmedetomidine hydrochloride fails to meet the requirement.

### SUMMARY

In a first aspect, the present disclosure provides a dexmedetomidine microneedle patch, including a substrate and a needle body disposed on a surface of the substrate, wherein the needle body includes dexmedetomidine hydrochloride, a matrix material, and a permeation enhancer; and
the permeation enhancer includes one or more of dodecyl-β-D-maltoside (DDM), ethylenediaminetetraacetic acid (EDTA), and tetrahydropiperine.

In an embodiment, the permeation enhancer includes either or both of ethylenediaminetetraacetic acid (EDTA) and tetrahydropiperine.

In an embodiment, in a composition of the needle body, a mass percentage of the permeation enhancer is in a range from 0.3% to 10%.

In an embodiment, in the composition of the needle body, the mass percentage of the permeation enhancer is in a range from 0.5% to 5%.

In an embodiment, in the composition of the needle body, the mass percentage of the permeation enhancer is in a range from 2% to 3%.

In an embodiment, the matrix material includes dextran.

In an embodiment, in the composition of the needle body, a mass percentage of dexmedetomidine hydrochloride is in a range from 4% to 56.5%; and/or
a mass percentage of the matrix material is in a range from 33.9% to 95.7%.

In an embodiment, the substrate is made of one or more of polyvinylpyrrolidone, polyvinyl alcohol, sodium hyaluronate, sodium carboxymethylcellulose, and hydroxypropyl methylcellulose.

In a second aspect, the present disclosure provides a method for preparing the dexmedetomidine microneedle patch in the first aspect, including:
mixing the matrix material, the permeation enhancer with water, and adding dexmedetomidine hydrochloride to prepare a needle body solution;
mixing and swelling a raw material of the substrate with a solvent to prepare a swollen substrate solution; and
molding and drying the needle body solution in a mold, adding the swollen substrate solution into the mold, and molding and drying the swollen substrate solution to obtain the substrate and the needle body, thereby preparing the dexmedetomidine microneedle patch.

In a third aspect, the present disclosure provides a method for treating insomnia in a subject in need thereof, including administering the dexmedetomidine microneedle patch in the first aspect to the subject.

### BRIEF DESCRIPTION OF THE DRAWINGS

FIG. 1 shows cumulative permeation rates of dexmedetomidine hydrochloride microneedle patches prepared in Examples and Comparative Examples at different time points.

FIG. 2 is a comparison of sleep latency time of respective administration groups in a test of dexmedetomidine hydrochloride microneedle prolonging sleep efficacy of pentobarbital sodium.

FIG. 3 is a comparison of sleep time of respective administration groups in the test of dexmedetomidine hydrochloride microneedle prolonging sleep efficacy of pentobarbital sodium.

### DETAILED DESCRIPTION

The dexmedetomidine microneedle patch of the present disclosure, and the preparation method and application thereof are further described in detail below in combination with specific embodiments. The present disclosure can be implemented in many different ways, and is not limited to the embodiments described herein. On the contrary, the purpose of providing these embodiments is to make the understanding of the disclosure of the present disclosure more thorough and comprehensive.

Unless otherwise defined, all technical and scientific terms used herein have the same meaning as commonly understood by those skilled in the art to which the present disclosure belongs. The terms used herein in the specification of the present disclosure are used only for the purpose of describing embodiments and are not intended to limit the present disclosure.

The terms “and/or”, “or/and”, and “as well as/or” used herein include both any one of two or more related listed items, and any or all combinations of related listed items. The any or all combinations include combinations of any two related listed items, combinations of any multiple related listed items, or a combination of all related listed items.

The “one or more” used herein refers to any one, or any two or more of the listed items.

In the present disclosure, “a first aspect”, “a second aspect”, “a third aspect”, etc. are only used for the purpose of description, and cannot be understood as indicating or implying relative importance or quantity, or implicitly indicating the importance or quantity of the indicated technical features. Moreover, “first”, “second”, “third”, etc. are only used for the purpose of non-exhaustive enumeration and description, and cannot be understood as constituting a closed-end limitation on quantity.

In the present disclosure, the technical features described in an open-type manner include both closed-ended technical solutions of the listed features, and open-ended technical solutions of the listed features.

In the present disclosure, unless otherwise specified, when a numerical range is referred to, the numerical range is continuous, and includes the minimum and maximum values of the range, as well as each value between the minimum value and the maximum value. Further, when the range refers to an integer range, it includes each integer between the minimum value and the maximum value of the range. In addition, when multiple ranges are provided to describe features or characteristics, the ranges can be combined. In other words, unless otherwise specified, all ranges disclosed herein are understood to include any or all subranges included therein.

Unless otherwise specified, the percentage content involved in the present disclosure indicates a mass percentage for solid-liquid mixing and solid-solid mixing, and a volume percentage for liquid-liquid mixing.

Unless otherwise specified, the percentage concentration involved in the present disclosure indicates a final concentration, which refers to the percentage of an added component in the system after the addition of the component.

The temperature parameter in the present disclosure, unless otherwise specified, is allowed to be either constant temperature treatment, or allowed to vary within a certain temperature range. The constant temperature treatment allows fluctuation of the temperature within an accuracy range controlled by the instrument.

In the present disclosure, the room temperature generally refers to 4° C. to 30° C., preferably 20±5° C.

The present disclosure provides a dexmedetomidine microneedle patch, a method for preparing the same, and application thereof. The dexmedetomidine microneedle patch exhibits relatively high cumulative drug permeation, which is beneficial to shortening the onset time of sleep-aiding effect, and prolonging the sleep-aiding effect time, thereby having good therapeutic efficacy for insomnia.

In some examples, the present disclosure provides a dexmedetomidine microneedle patch, including a substrate and a needle body disposed on a surface of the substrate, wherein the needle body includes dexmedetomidine hydrochloride, a matrix material, and a permeation enhancer; and
the permeation enhancer includes one or more of dodecyl-β-D-maltoside (DDM), ethylenediaminetetraacetic acid (EDTA), and tetrahydropiperine.

In some examples, the permeation enhancer includes either or both of ethylenediaminetetraacetic acid (EDTA) and tetrahydropiperine. By using a suitable permeation enhancer, a relatively high cumulative drug permeation can be achieved with a small dosage, thereby obtaining a better therapeutic effect on insomnia.

In some examples, in a composition of the needle body, a mass percentage of the permeation enhancer is in a range from 0.3% to 10%. By reasonably controlling the mass percentage of the permeation enhancer, a relatively high cumulative drug permeation can be achieved, thereby obtaining a better therapeutic effect on insomnia. Specifically, in the composition of the needle body, the mass percentage of the permeation enhancer includes but is not limited to: 0.3%, 0.5%, 0.6%, 0.8%, 1.0%, 1.2%, 1.5%, 1.8%, 2%, 2.4%, 3%, 3.5%, 4%, 4.8%, 6%, 8%, 9.6%, 10%, or a range between any two of the above values. Further, in the composition of the needle body, the mass percentage of the permeation enhancer is in a range from 0.5% to 5%. Further, in the composition of the needle body, the mass percentage of the permeation enhancer is in a range from 2% to 3%.

In some examples, the matrix material includes dextran. Suitable matrix material can enable the drug to be dissolved quickly and released in the skin, can concentrate the drug at the needle tip to reduce drug migration, and improve the delivery efficiency and utilization of the drug.

In some examples, a mass percentage of the matrix material is in a range from 33.9% to 95.7%. Specifically, in the composition of the needle body, the mass percentage of the matrix material includes but is not limited to: 33.9%, 40%, 45%, 50%, 55%, 60%, 65%, 70%, 75%, 80%, 82%, 85%, 88%, 90%, 91%, 92.5%, 95.7%, or a range between any two of the above values. Further, the mass percentage of the matrix material is in a range from 80% to 92.5%.

In some examples, in the composition of the needle body, a mass percentage of dexmedetomidine hydrochloride is in a range from 4% to 56.5%. Specifically, in the composition of the needle body, the mass percentage of dexmedetomidine hydrochloride includes but is not limited to: 4%, 4.5%, 5%, 5.5%, 6%, 6.5%, 7%, 7.5%, 8%, 8.5%, 9%, 9.5%, 10%, 20%, 25%, 30%, 35%, 40%, 45%, 50%, 56.5%, or a range between any two of the above values. Further, in the composition of the needle body, the mass percentage of dexmedetomidine hydrochloride is in a range from 7% to 10%.

In some examples, the substrate is made of one or more of polyvinylpyrrolidone, polyvinyl alcohol, sodium hyaluronate, sodium carboxymethylcellulose, and hydroxypropyl methylcellulose. Without any limitation, for example, the polyvinyl pyrrolidone can be one or more of PVP K90, PVP K30, and PVP K60.

In some other examples, the present disclosure provides a method for preparing the dexmedetomidine microneedle patch as described above, including the following steps:
mixing the matrix material, the permeation enhancer with water, and adding dexmedetomidine hydrochloride to prepare a needle body solution;
mixing and swelling a raw material of the substrate with a solvent to prepare a swollen substrate solution; and
molding and drying the needle body solution in a mold, adding the swollen substrate solution into the mold, and molding and drying the swollen substrate solution to obtain the substrate and the needle body, thereby preparing the dexmedetomidine microneedle patch.

In some examples, in the preparation of the swollen substrate solution, the solvent includes anhydrous ethanol.

In some examples, the step of molding the needle body solution in the mold includes:
adding the needle body solution to a microneedle negative mold, and performing centrifugation at 3500 rpm to 4500 rpm at 0° C. to 10° C. for 5 mins to 15 mins;
turning the mold over by 180 degrees, and performing centrifugation again at 3500 rpm to 4500 rpm at 0° C. to 10° C. for 5 mins to 15 mins; and
removing the excess needle body solution, and performing centrifugation again at 3500 rpm to 4500 rpm at 0° C. to 10° C. for 25 mins to 35 mins.

In some examples, the step of adding the swollen substrate solution into the mold and molding the swollen substrate solution includes:
adding the swollen substrate solution into the microneedle negative mold containing the dried needle body, performing centrifugation at 3500 rpm to 4500 rpm at 0° C. to 10° C. for 1 min to 10 mins, performing a vacuuming treatment, and performing centrifugation again at 3500 rpm to 4500 rpm at 0° C. to 10° C. for 1 min to 10 mins.

It can be understood that, after the needle body and the substrate are formed, the dexmedetomidine microneedle patch can be prepared by demolding.

In some other examples, the present disclosure further provides a method for treating insomnia in a subject in need thereof, including administering the dexmedetomidine microneedle patch as described above to the subject. Without any limitation, treating insomnia means shortening sleep latency and/or prolonging sleep time.

In the above-mentioned dexmedetomidine microneedle patch, in the presence of dexmedetomidine hydrochloride and the matrix material, one or more of dodecyl-β-D-maltoside (DDM), ethylenediaminetetraacetic acid (EDTA), and tetrahydropiperine is incorporated as the permeation enhancer, which can significantly improve the drug transdermal delivery efficiency of dexmedetomidine hydrochloride, promote release of the drug into the blood, and significantly increase the cumulative skin permeation of dexmedetomidine hydrochloride in the microneedle, thereby achieving a good therapeutic effect on insomnia, shortening the onset time of sleep-aiding effect, and prolonging the sleep-aiding effect time.

Meanwhile, it is found in study that compared with a dexmedetomidine hydrochloride injection, the above-mentioned dexmedetomidine microneedle patch can achieve a shorter sleep latency and longer sleep time, indicating that the addition of the permeation enhancer promotes the treatment effect of dexmedetomidine on insomnia, with a synergistic effect.

In addition, compared with conventional insomnia treatment drugs, the above-mentioned dexmedetomidine microneedle patch has the following advantages.
(1) The dexmedetomidine hydrochloride microneedle patch exhibits a high bioavailability.
(2) There is no need to combine an additional drug for administration, especially those with adverse effects such as addiction, dependence, hangover effects, etc., thereby improving the safety of medication.
(3) The dexmedetomidine microneedle patch is administered via a transdermal route, which is convenient for patients to administer the medicine in familiar places outside of medical facilities, and thus is convenient to carry and has good administration convenience. Meanwhile, compared with conventional dexmedetomidine injection and nasal spray, the dexmedetomidine microneedle patch reduces the pain caused by injection or the irritation caused by nasal administration, thereby improving medication compliance for patients, and exhibiting better clinical application value.

For experimental parameters not specified in the following specific examples, reference is preferentially made to the instructions given in the present disclosure, and reference may also be made to experimental manuals in the art or other experimental methods known in the art or to experimental conditions recommended by manufacturers.

The raw materials and reagents involved in the following specific examples can be obtained from commercial sources, or can be prepared by those skilled in the art according to known methods.

The dexmedetomidine microneedle patches in Examples and Comparative Examples are prepared as follows:
(1) Preparation of a needle body solution: The matrix material and the permeation enhancer in corresponding proportions were weighed, ultrapure water was added, the mixture was stirred to dissolve to obtain an auxiliary material solution. Dexmedetomidine hydrochloride was weighed, and added to the auxiliary material solution. The resulting mixture was stirred to dissolve to obtain a needle body solution. In Comparative Example 1, no permeation enhancer was added.
(2) Preparation of a substrate solution: 17.5 g of PVP K90 was weighted and added into a centrifuge cup, and 100 mL of anhydrous ethanol was added. The mixture was mixed thoroughly, and left overnight to fully swelling to obtain a substrate solution.
(3) Preparation of needle bodies: 200 μL of the needle body solution prepared in (1) was taken and added to a microneedle negative mold, and centrifuged at 4000 rpm at 0° C. to 10° C. for 10 mins. After centrifugation, the mold was turned over by 180 degrees, and centrifuged again at 4000 rpm at 0° C. to 10° C. for 10 mins. Then, the microneedle mold was taken out, the excess needle body solution on the upper layer was scraped off, and centrifuged again at 4000 rpm at 0° C. to 10° C. for 30 mins. The microneedle mold was placed in a moisture-proof cabinet to dry the needle bodies.
(4) Preparation of a substrate: 300 μL of the substrate solution prepared in (2) was taken and added into the microneedle negative mold containing the dried needle bodies, and centrifuged at 4000 rpm at 0° C. to 10° C. for 3 mins to evenly disperse the substrate solution on the mold. Then, the microneedle mold was placed in a vacuum drying cabinet, vacuumized to reach a vacuum degree close to −0.1 Mpa, and immediately the air valve was opened to restore the vacuum drying cabinet to a normal pressure. This process was repeated twice. The mold was taken out, placed in a centrifuge, and centrifuged at 4000 rpm at 0° C. to 10° C. for 5 mins to remove bubbles on the surface of the substrate. The mold was taken out, and dried in an oven at 40° C. for 12 hours.
(5) Demolding: The microneedles were peeled off from the mold to obtain the soluble dexmedetomidine hydrochloride microneedle patch.

### Comparative Example 1

In the comparative example, a dexmedetomidine microneedle patch consisted of a substrate and a needle body disposed on the surface of the substrate was provided.

The substrate was made of polyvinyl pyrrolidone, i.e., PVP K90.

The components of the needle body are shown in Table 1 below.

TABLE 1

| Component | Mass percentage |
|---|---|
| Dexmedetomidine hydrochloride | 9.1% |
| Dextran 40 | 90.9% |

### Comparative Example 2

In the comparative example, a dexmedetomidine microneedle patch consisted of a substrate and a needle body disposed on the surface of the substrate was provided.

The substrate was made of polyvinyl pyrrolidone, i.e., PVP K90.

The components of the needle body are shown in Table 2 below.

TABLE 2

| Component | Mass percentage |
|---|---|
| Dexmedetomidine hydrochloride | 9.1% |
| Dextran 40 | 86.1% |
| Hyaluronidase | 4.8% |

### Example 1

In the example, a dexmedetomidine microneedle patch consisted of a substrate and a needle body disposed on the surface of the substrate was provided.

The substrate was made of polyvinyl pyrrolidone, i.e., PVP K90.

The components of the needle body are shown in Table 3 below.

TABLE 3

| Component | Mass percentage |
|---|---|
| Dexmedetomidine hydrochloride | 9.1% |
| Dextran 40 | 90.6% |
| Dodecyl-β-D-maltoside (DDM) | 0.3% |

### Example 2

In the example, a dexmedetomidine microneedle patch consisted of a substrate and a needle body disposed on the surface of the substrate was provided.

The substrate was made of polyvinyl pyrrolidone, i.e., PVP K90.

The components of the needle body are shown in Table 4 below.

TABLE 4

| Component | Mass percentage |
|---|---|
| Dexmedetomidine hydrochloride | 9.1% |
| Dextran 40 | 90.3% |
| Dodecyl-β-D-maltoside (DDM) | 0.6% |

### Example 3

In the example, a dexmedetomidine microneedle patch consisted of a substrate and a needle body disposed on the surface of the substrate was provided.

The substrate was made of polyvinyl pyrrolidone, i.e., PVP K90.

The components of the needle body are shown in Table 5 below.

TABLE 5

| Component | Mass percentage |
|---|---|
| Dexmedetomidine hydrochloride | 9.1% |
| Dextran 40 | 89.7% |
| Dodecyl-β-D-maltoside (DDM) | 1.2% |

### Example 4

In the example, a dexmedetomidine microneedle patch consisted of a substrate and a needle body disposed on the surface of the substrate was provided.

The substrate was made of polyvinyl pyrrolidone, i.e., PVP K90.

The components of the needle body are shown in Table 6 below.

TABLE 6

| Component | Mass percentage |
|---|---|
| Dexmedetomidine hydrochloride | 9.1% |
| Dextran 40 | 88.5% |
| Dodecyl-β-D-maltoside (DDM) | 2.4% |

### Example 5

In the example, a dexmedetomidine microneedle patch consisted of a substrate and a needle body disposed on the surface of the substrate was provided.

The substrate was made of polyvinyl pyrrolidone, i.e., PVP K90.

The components of the needle body are shown in Table 7 below.

TABLE 7

| Component | Mass percentage |
|---|---|
| Dexmedetomidine hydrochloride | 9.1% |
| Dextran 40 | 86.1% |
| Dodecyl-β-D-maltoside (DDM) | 4.8% |

### Example 6

In the example, a dexmedetomidine microneedle patch consisted of a substrate and a needle body disposed on the surface of the substrate was provided.

The substrate was made of polyvinyl pyrrolidone, i.e., PVP K90.

The components of the needle body are shown in Table 8 below.

TABLE 8

| Component | Mass percentage |
|---|---|
| Dexmedetomidine hydrochloride | 9.1% |
| Dextran 40 | 81.3% |
| Dodecyl-β-D-maltoside (DDM) | 9.6% |

### Example 7

In the example, a dexmedetomidine microneedle patch consisted of a substrate and a needle body disposed on the surface of the substrate was provided.

The substrate was made of polyvinyl pyrrolidone, i.e., PVP K90.

The components of the needle body are shown in Table 9 below.

TABLE 9

| Component | Mass percentage |
|---|---|
| Dexmedetomidine hydrochloride | 9.1% |
| Dextran 40 | 90.4% |
| Ethylenediaminetetraacetic acid (EDTA) | 0.5% |

### Example 8

In the example, a dexmedetomidine microneedle patch consisted of a substrate and a needle body disposed on the surface of the substrate was provided.

The substrate was made of polyvinyl pyrrolidone, i.e., PVP K90.

The components of the needle body are shown in Table 10 below.

TABLE 10

| Component | Mass percentage |
|---|---|
| Dexmedetomidine hydrochloride | 9.1% |
| Dextran 40 | 89.9% |
| Tetrahydropiperine | 1.0% |

### Test Example

### (1) In Vitro Transdermal Study

### Experimental Method:

A pigskin was fixed on a storage table directly below a probe of a tensile tester, and the microneedle patch was fixed on the probe of the tensile tester with the needle tip facing downward.

The parameters of the tensile tester were set to maintain the drug administration state for 3 minutes under a force of 100 N. After the drug administration, the pigskin was transferred to a Franz diffusion cell for drug transdermal study. Samples were taken at 1 hour, 2 hours, 6 hours, 16 hours, and 24 hours respectively to detect the drug content, and the cumulative release amount was calculated.

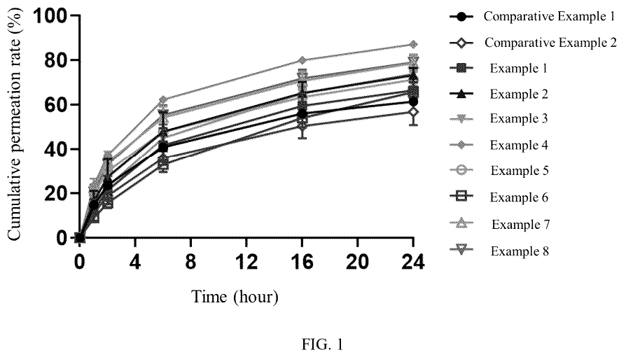

At each time point, the drug content was tested three times in parallel. The 24 h cumulative permeation rates of the drugs are shown in Table 11 below, and the cumulative permeation rates at respective time points are shown in FIG. 1.

TABLE 11

| | | | 24 h |
|---|---|---|---|
| | Kind of | | cumulative |
| | permeation | Concentration | drug permeation |
| | enhancer | (%) | rate (%) |
| Comparative | Without permeation | / | 61.4 |
| Example 1 | enhancer | | |
| Comparative | Hyaluronidase | 4.8 | 56.8 |
| Example 2 | | | |
| Example 1 | DDM | 0.3 | 66.4 |
| Example 2 | | 0.6 | 73.2 |
| Example 3 | | 1.2 | 73.9 |
| Example 4 | | 2.4 | 87.1 |
| Example 5 | | 4.8 | 71.2 |
| Example 6 | | 9.6 | 65.6 |
| Example 7 | EDTA | 0.5 | 78.6 |
| Example 8 | Tetrahydropiperine | 1.0 | 79.1 |

### (2) Test of Dexmedetomidine Hydrochloride Microneedle Prolonging Sleep Efficacy of Pentobarbital Sodium.

### 2.1 Test Preparation

Reagents: 0.9% sodium chloride solution, 1.5% pentobarbital sodium, dexmedetomidine hydrochloride microneedle of Example 4, commercially available dexmedetomidine hydrochloride injection (100 μg/mL).

Test animals: 20 male SD rats, SPF grade, weighing 180 g to 200 g.

### 2.2 Test Methods

The animals were randomly divided into 4 groups according to their body weight, i.e., a normal control group, a dexmedetomidine hydrochloride microneedle low dose group (4 μg/rat), a dexmedetomidine hydrochloride microneedle high dose group (6 μg/rat), and a dexmedetomidine hydrochloride injection group (4 μg/rat).

### 2.3 the Test Scheme is Shown in Table 12.

TABLE 12

| | Drug of | Route of | Dosage of | Frequency of |
|---|---|---|---|---|
| Group | administration | administration | administration | administration |
| Normal control | 0.9% sodium | i.v. | 4 μg/rat | Single dose |
| group | chloride solution | | | |
| Dexmedetomidine | dexmedetomidine | TDDs | 4 μg/rat | Single dose |
| hydrochloride | hydrochloride | | | |
| microneedle low | | | | |
| dose group | | | | |
| Dexmedetomidine | dexmedetomidine | TDDs | 6 μg/rat | Single dose |
| hydrochloride | hydrochloride | | | |
| microneedle high | | | | |
| dose group | | | | |
| Dexmedetomidine | dexmedetomidine | i.v. | 4 μg/rat | Single dose |
| hydrochloride | hydrochloride | | | |
| injection group | | | | |

Note:
i.v. is short for intravenous injection; TDDs is short for transdermal administration.

According to the results of previous experiments, after the rats were intraperitoneally injected with 30 mg/kg pentobarbital sodium, the righting reflex of the rat disappeared in about 10 minutes. Therefore, the suprathreshold dose of pentobarbital sodium in rats was set to be 30 mg/kg. The drugs in respective doses were given to rats according to Table 12, and immediately after administration, the pentobarbital sodium with 30 mg/kg of suprathreshold dose was injected intraperitoneally. The time of injection of pentobarbital sodium, the time of disappearance of righting reflex, and the time of recovery of righting reflex were recorded. The disappearance of bilateral righting reflex of rats for 60 seconds was used as the standard for falling asleep. The sleep latency and sleep time were calculated respectively.

Sleep latency=the time of disappearance of righting reflex−the time of injection of pentobarbital sodium

Sleep time=the time of recovery of righting reflex−the time of disappearance of righting reflex

### 2.4 Data Analysis

The time of injection of pentobarbital sodium, the time of disappearance of righting reflex, and the time of recovery of righting reflex were entered into Excel software, and the sleep latency and the sleep time were calculated. Drawings were made with GraphPad Prism8 software. Statistical analysis and regression calculation of rat sleep latency ED 50 were made with SPSS software. The sleep latency and sleep time of each dose group were tested for variance homogeneity. If the variance was homogeneous (i.e., p>0.05), a one-way analysis of variance was performed.

If there is a significant difference (i.e., p≤0.05), a Dunnett test will be performed between each dose group and the normal control group; otherwise, the test will be terminated. If the variance is unequal (i.e., p≤0.05), a non-parametric test (Kruskal-Wallis H test, i.e., K-W H test) will be performed. If the K-W H test shows a statistical difference (i.e., p≤0.05), a Mann-Whitney U test will be performed between each dose group and the control group; otherwise, the test will be terminated.

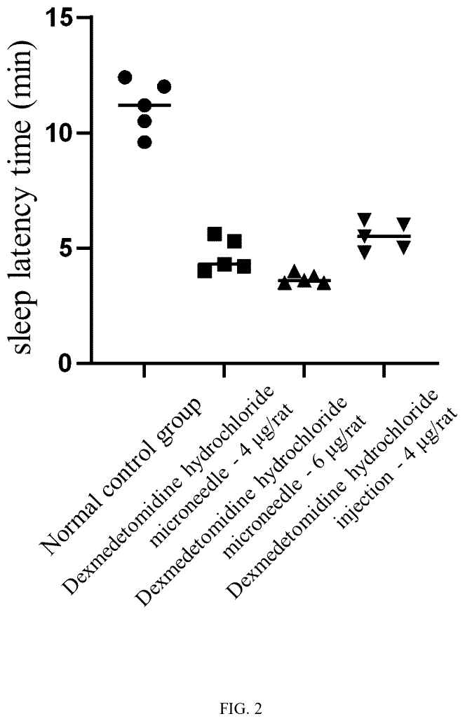

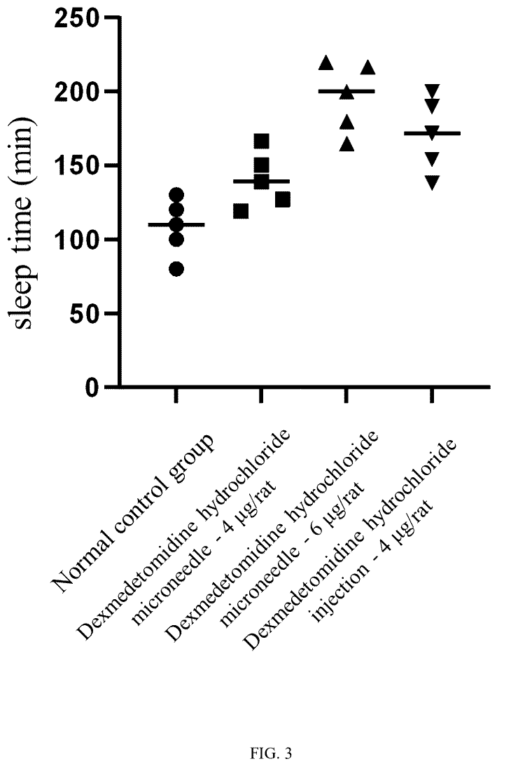

### 2.5 the Test Results are Shown in Table 13 and FIGS. 2 and 3.

TABLE 13
Effects of various dexmedetomidine hydrochloride
formulations on the hypnotic effect of pentobarbital
sodium in suprathreshold dose (Mean ± SD)

| | sleep | sleep |
|---|---|---|
| | latency | time |
| Group | time (min) | (min) |
| Normal control group | 11.4 ± 1.13 | 108.0 ± 19.24 |
| Dexmedetomidine | 4.68 ± 0.72**** | 140.4 ± 18.97 |
| hydrochloride | | |
| microneedle | | |
| low dose group | | |
| Dexmedetomidine | 3.68 ± 0.22****## | 196.4 ± 23.71**** |
| hydrochloride | | |
| microneedle | | |
| high dose group | | |
| Dexmedetomidine | 5.5 ± 0.61**** | 170.8 ± 25.24*** |
| hydrochloride injection | | |
| group | | |

Note:
Compared with the normal control group, ***P < 0.001, ****P < 0.0001; compared with dexmedetomidine hydrochloride injection, ##P < 0.01.

It can be seen that, after intraperitoneal injection of 30 mg/kg pentobarbital sodium, the sleep latency of normal rats was about 11.4 min. The dexmedetomidine hydrochloride microneedles (4 μg/rat and 6 μg/rat) shortened the sleep latency of rats in a dose-dependent manner, which was significantly different from the solvent control group (P<0.0001). At the same dose, the dexmedetomidine hydrochloride microneedle exhibited equivalent effect on shortening the sleep latency to that of the dexmedetomidine hydrochloride injection. At a dose of 6 μg/rat, the dexmedetomidine hydrochloride microneedle can significantly shorten the sleep latency of rats, which is even significantly better than that of the dexmedetomidine hydrochloride injection at a dose of 4 μg/rat (P<0.01).

After intraperitoneal injection of 30 mg/kg pentobarbital sodium, the sleep time of normal rats was about 108 min. The dexmedetomidine hydrochloride microneedles (4 μg/rat and 6 μg/rat) prolonged the sleep time of rats in a dose-dependent manner, which was significantly different from the solvent control group (P<0.0001). At the same dose, the dexmedetomidine hydrochloride microneedle exhibited equivalent effect on prolonging the sleep time to that of the dexmedetomidine hydrochloride injection. At a dose of 6 μg/rat, the dexmedetomidine hydrochloride microneedle can significantly prolong the sleep time of rats, which is slightly better than that of the dexmedetomidine hydrochloride injection at a dose of 4 μg/rat.

The technical features of the above-mentioned embodiments can be combined arbitrarily. In order to make the description concise, not all possible combinations of the technical features are described in the embodiments. However, as long as there is no contradiction in the combination of these technical features, the combinations should be considered as in the scope of the present disclosure.

The above-described embodiments are only several implementations of the present disclosure, and the descriptions are relatively specific and detailed, but they should not be construed as limiting the scope of the present disclosure. It should be understood by those of ordinary skill in the art that various modifications and improvements can be made without departing from the concept of the present disclosure, and all fall within the protection scope of the present disclosure. It should be understood that technical solutions obtained by those of ordinary skill in the art through logical analysis, reasoning, or limited experiments based on the technical solutions provided in the present disclosure are all within the protection scope of the appended claims of the present disclosure. Therefore, the patent protection of the present disclosure shall be defined by the appended claims, and the specification can be used to explain the content of the claims.

1. A dexmedetomidine microneedle patch, comprising a substrate and a needle body disposed on a surface of the substrate, wherein the needle body comprises dexmedetomidine hydrochloride, a matrix material, and a permeation enhancer; and
the permeation enhancer comprises one or more of dodecyl-β-D-maltoside, ethylenediaminetetraacetic acid, and tetrahydropiperine.

2. The dexmedetomidine microneedle patch according to claim 1, wherein the permeation enhancer comprises either or both of ethylenediaminetetraacetic acid and tetrahydropiperine.

3. The dexmedetomidine microneedle patch according to claim 1, wherein in a composition of the needle body, a mass percentage of the permeation enhancer is in a range from 0.3% to 10%.

4. The dexmedetomidine microneedle patch according to claim 3, wherein in the composition of the needle body, the mass percentage of the permeation enhancer is in a range from 0.5% to 5%.

5. The dexmedetomidine microneedle patch according to claim 4, wherein in the composition of the needle body, the mass percentage of the permeation enhancer is in a range from 2% to 3%.

6. The dexmedetomidine microneedle patch according to claim 1, wherein the matrix material comprises dextran.

7. The dexmedetomidine microneedle patch according to claim 1, wherein in a composition of the needle body, a mass percentage of dexmedetomidine hydrochloride is in a range from 4% to 56.5%.

8. The dexmedetomidine microneedle patch according to claim 1, wherein in a composition of the needle body, a mass percentage of the matrix material is in a range from 33.9% to 95.7%.

9. The dexmedetomidine microneedle patch according to claim 1, wherein the substrate is made of one or more of polyvinylpyrrolidone, polyvinyl alcohol, sodium hyaluronate, sodium carboxymethylcellulose, and hydroxypropyl methylcellulose.

10. A method for preparing the dexmedetomidine microneedle patch according to claim 1, comprising:
mixing the matrix material, the permeation enhancer with water, and adding dexmedetomidine hydrochloride to prepare a needle body solution;
mixing and swelling a raw material of the substrate with a solvent to prepare a swollen substrate solution; and
molding and drying the needle body solution in a mold, adding the swollen substrate solution into the mold, and molding and drying the swollen substrate solution to obtain the substrate and the needle body, thereby preparing the dexmedetomidine microneedle patch.

11. A method for treating insomnia in a subject in need thereof, comprising administering the dexmedetomidine microneedle patch according to claim 1 to the subject.
